(19) United States
(12) Patent Application Publication
(10) Pub. No.: US 2009/0175905 A1
(43) Pub. Date: Jul. 9, 2009

(54) TRANSFECTION REAGENT AND METHOD FOR ENHANCING TRANSFECTION EFFICIENCY

(75) Inventors: Wen-Chi Tseng, Taipei (TW); Tsuei-Yun Fang, Taipei (TW); Chien-Hsiang Tang, Taipei (TW)

Correspondence Address: JIANQ CHYUN INTELLECTUAL PROPERTY OFFICE, 7 FLOOR-1, NO. 100, ROOSEVELT ROAD, SECTION 2, TAIPEI, 100, TW

(73) Assignee: National Taiwan University of Science and Technology, Taipei (TW)

(21) Appl. No.: 12/168,071

(22) Filed: Jul. 4, 2008

(30) Foreign Application Priority Data
Jan. 4, 2008 (TW) 97100328

(51) Int. Cl. A61K 39/12; A61K 31/7088; A61K 39/00

(52) U.S. Cl. 424/205.1; 514/44; 424/184.1

(57) ABSTRACT

A transfection reagent and a method for enhancing transfection efficiency are described. A nucleic acid is provided, and a disaccharide is added as a transfection reagent. The disaccharide is composed of two identical monosaccharides. The transfection reagent is then applied to cells to introduce the nucleic acid into the cells.

### CROSS-REFERENCE TO RELATED APPLICATION

This application claims the priority benefit of Taiwan application serial no. 97100328, filed on Jan. 4, 2008. The entirety of the above-mentioned patent application is hereby incorporated by reference herein and made a part of specification.

### BACKGROUND OF THE INVENTION

1. Field of Invention

The present invention relates to a transfection reagent and a method for enhancing transfection efficiency. More particularly, the present invention relates to a transfection reagent incorporated with a disaccharide and a method for enhancing transfection efficiency.

2. Description of Related Art

As the discovery of gene and the rapid development of genetics, foreign gene can be used to alter the characteristics of cells and to further change the gene expression of a biological entity by applying genetic techniques. Due to the development of biotechnology, genetic materials, such as deoxyribonucleic acid (DNA), can be transferred into a biological entity to alter its characteristics. This type of biotechnology has been broadly applied in basic research and in the modification of agricultural products. Recently, the technology of gene transfer, such as gene therapy and nucleic acid vaccine, has been applied in the treatment and therapy of animal diseases. A successful application of transfer of genetic materials in the prophylaxis and medical therapy would greatly advance medical treatments on many genetic-related and immunology-related diseases.

In general, there are various methods for gene transfection, and one of the earliest approaches includes using bacteria or virus as a vector to transfer gene into plant cells. After a long term development and modification, the method of gene transfection has become more stable and the transfection efficiency of gene has improved noticeably. However using bacteria or virus as a vector may generate the side effects of nonspecific immune response or gene reorganization. Hence, the application of this method greatly increases risks and hampers safety.

Minimizing the side effects of using viral vectors can be achieved by the application of non-viral vectors. Currently, the design of non-viral vectors general utilizes chemical synthesis to form new type of lipids and of polymers or modifies the existing vectors to enhance the transfection efficiency. However, these new type of carriers formed by chemical synthesis normally require subsequent purification and characterization before being able to apply to cells or animals in the transfection experiments. Hence, the conventional approach is inconvenient and is not readily accessible. Moreover, the application of the newly synthesized vectors or modified vectors is limited. The newly synthesized compounds may be effectively applied in vitro, but turn out to be much less effective in vivo. Similarly, the newly synthesized compounds may be effectively applied in vivo, but have much lower effectiveness in vitro. In other words, a vector formed by chemical synthesis or modification hardly enhances the efficiency in both in vivo and in vitro transfection. Furthermore, when compared with a viral vector, the transfection efficiency of a non-viral vector is normally less desirable.

Recently, gene transfection methods, for example, electroporation, microinjection, gene gun, etc., which are based on physical theory or mechanical theory have been developed. Since the application of bacteria or virus can be precluded, invoking side effects and safety risks can be reduced. Hence, these methods are more suitable to the field of medical treatment and therapy. However, the stability and success rate of these types of physical methods are rather low, and thereby can not be broadly applied.

Accordingly, to enhance the efficiency in both in vivo transfection and in vitro transfection and to decrease the cytotoxicity concurrently becomes one of the criteria in designing new nonviral vectors.

### SUMMARY OF THE INVENTION

The present invention is to provide a transfection reagent, in which the transfection efficiency is enhanced and the amount of nucleic acid being used is reduced. Further, the duration for the nucleic acid to remain in the cell increases.

The present invention is to provide a method for increasing transfection efficiency, wherein the method is applicable in in vivo and in vitro.

The present invention is to provide a transfection reagent including a nucleic acid and a disaccharide, wherein the disaccharide is formed with two identical monosaccharide units.

According to one embodiment of the present invention, the disaccharide includes, for example, trehalose or cellobiose.

According to one embodiment of the present invention, the transfection reagent includes a vector.

According to one embodiment of the present invention, the vector includes, for example, a viral vector or a non-viral vector.

According to one embodiment of the present invention, the non-viral vector includes but not limited to liposomes or polymers.

According to one embodiment of the present invention, when the non-viral vector is liposomes, the disaccharide is trehalose or cellobiose.

According to one embodiment of the present invention, when the non-viral vector is a polymer, the disaccharide is trehalose.

According to one embodiment of the present invention, the above-mentioned transfection reagent is applicable in an electroporation system or a naked plasmid delivery system.

According to one embodiment of the present invention, the above-mentioned transfection reagent is applicable in an in vitro cell culture transfection system or an in vivo drug delivery system.

According to one embodiment of the present invention, when the above transfection reagent is applied in an in vitro cell culture transfection system, the concentration of disaccharide in the transfection reagent is between about 10 mM to 300 mM.

According to one embodiment of the present invention, when the above transfection reagent is used in an in vivo drug delivery system, the concentration of disaccharide is between about 15 mg/kg body weight to about 1000 mg/kg body weight.

The present invention provides another method of enhancing transfection efficiency. A nucleic acid is provided. A disaccharide is then incorporated with the nucleic acid as a transfection reagent, wherein the disaccharide is formed with two identical monosaccharide units. Thereafter, the transfection reagent is applied to cells to transfect the nucleic acid into the cells.

According to one embodiment of the present invention, the disaccharide includes trehalose or cellobiose, for example.

According to one embodiment of the present invention, the transfection reagent includes a vector.

According to one embodiment of the present invention, the vector includes, for example, a viral vector or a non-viral vector.

According to one embodiment of the present invention, the non-viral vector includes, but not limited to, liposomes or polymers.

According to one embodiment of the present invention, when the non-viral vector is liposomes, the disaccharide, but not limited to, trehalose or cellobiose.

According to one embodiment of the present invention, when the non-viral vector is a polymer, the disaccharide, but not limited to, trehalose.

According to one embodiment of the present invention, the above-mentioned transfection reagent is applicable in an electroporation system or a naked plasmid delivery system.

According to one embodiment of the present invention, the above cells include in vitro cells or an in vivo cells.

According to one embodiment of the present invention, when the above transfection reagent is used in an in vitro cell culture transfection system, the concentration of disaccharide in the transfection reagent is between about 10 mM to 300 mM.

According to one embodiment of the present invention, when the above transfection reagent is used in an in vivo drug delivery system, the concentration of disaccharide is between about 15 mg/kg body weight to about 1000 mg/kg body weight.

According to the transfection reagent and a method of enhancing transfection efficiency of the present invention, by incorporating a disaccharide with the to-be-transfected nucleic acid as a transfection reagent, both the in vitro transfection efficiency and the in vivo transfection efficiency can be improved and the time for the nucleic acid remaining inside the cell can be extended. The transfection efficiency is desirable even when the amount of nucleic acid used is lowered.

Moreover, the transfection reagent and the method of enhancing transfection efficiency are applicable in various transfection systems and various types of vectors. According to the present invention, by incorporating a disaccharide into a transfection reagent, the transfection efficiency is enhanced. Further, while the transfection efficiency is enhanced, the toxic side effects to the transfected cells by the transfection reagent can be reduced.

In order to achieve the aforementioned and other objects, the features and advantages of the present invention comprehensible, a preferred embodiment accompanied with figures is described in detail below.

### BRIEF DESCRIPTION OF THE DRAWINGS

FIG. 1 is a flowchart showing the method of enhancing transfection efficiency according to one embodiment of the present invention.

FIG. 2 presents the effects of disaccharides on the transgene expression of cultured Chinese hamster ovary (CHO) cells subjected to an in vitro transfection of DNA with a transfection reagent containing DOTAP and various disaccharides.

FIGS. 3A and 3B present the effects of disaccharides on the transgene expression of cultured Chinese hamster ovary (CHO) cells subjected to an in vitro transfection of DNA with a transfection reagent containing different lipid-based vectors plus different disaccharides.

FIG. 4A presents the effects of disaccharides on the transgene expression after intravenous injection of DOTAP-based vectors with various disaccharides by way of mouse tail vein.

FIG. 4B presents the effects of disaccharides on the transgene expression after intravenous injection of different lipid-based vectors with various disaccharides by way of mouse tail vein.

FIG. 5 presents the effects of disaccharides on the cellular cytotoxicity of the in vitro cultured CHO cells transfected with transfection reagents containing lipid-based carriers.

FIG. 6 presents the effects of disaccharides on the transfection efficiency of in vitro cultured CHO cells transfected with transfection reagents containing PEI and different disaccharides.

FIGS. 7A and & 7B show the optimal trehalose concentrations for enhancing the transfection expression mediated by DNA-PEI complexes in different cell lines.

FIG. 8 presents the effects of trehalose on the cellular cytotoxicity of the in vitro cultured CHO cells transfected with transfection reagents containing DNA-PEI complexes.

FIG. 9 presents the effects of various disaccharides on the transgene expression of mouse muscles after direct intramuscular injection of naked plasmid.

FIG. 10 presents the effects of trehalose concentrations on the transgene expression of mouse muscles after direct intramuscular injection of naked plasmid.

Experiment 11 presents the effects of gene dosage on the transgene expression of mouse muscles in the presence of trehalose after direct intramuscular injection of naked plasmid.

FIG. 12 presents the effects of the trehalose on the durations of transgene expression of mouse muscles after direct intramuscular injection of naked plasmid.

### DESCRIPTION OF EMBODIMENTS

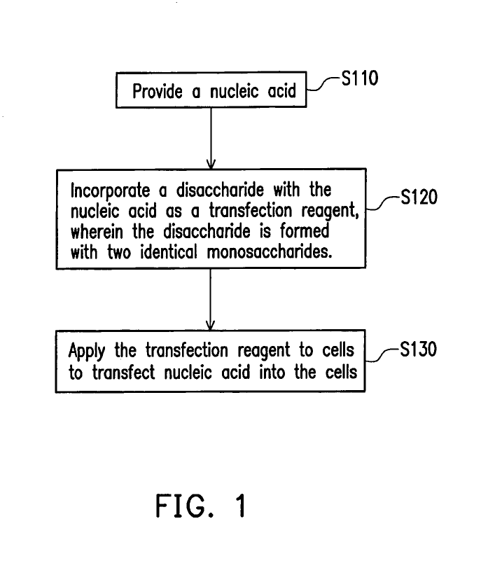

FIG. 1 is a flowchart showing the method of enhancing transfection efficiency according to one embodiment of the present invention.

Referring to FIG. 1, in step S110, a nucleic acid is provided. The nucleic acid includes any type of nucleic acids that can be transfected into a cell based on the existing transfection technology. The nucleic acid includes, for example, deoxyribonucleic acid (DNA), ribonucleic acid (RNA), or peptide nucleic acid (PNA). Further, the nucleic acid of the invention may include plasmid DNA, triple-stranded DNA, small interfering RNA (siRNA), etc.

Thereafter, in step S120, a disaccharide is incorporated with the nucleic acid to form a transfection reagent. Since a disaccharide molecule is formed by a condensation reaction with two monosaccharide molecules and a loss of a water molecule, the disaccharide molecule can be a homo-disaccharide or a hetero-disaccharide. The disaccharide in step S120 includes, for example, a disaccharide formed with two identical monosaccharide units. In one embodiment of the invention, the disaccharide in the transfection reagent of the invention includes, trehalose or cellobiose, which is formed with two glucose molecules, for example. Trehalose is one type of non-reducing disaccharide formed with two glucose molecules by a α(1→1)α bond. Trehalose can serve as a stabilizer of protein and is employed to preserve animal cells and platelets. Hence, the incorporation of trehalose into a transfection reagent enhances the stability of gene in the transfection reagent. Moreover, trehalose can suppress the fusion between liposomes and the fusion between phagosomes and lysosomes. Hence, the disintegration of plasmid can be prevented and the level of transgenic expression can be enhanced. Cellobiose is one type of disaccharide formed with two glucose molecules by a β(1→4) bond. Similar to trehalose, cellobiose is also capable of gene stabilization. Hence, transfection efficiency can be improved. Moreover, both trehalose and cellobiose are non-toxic materials. Therefore, no toxic effect will be induced to the cells when trehalose and cellobiose are used as a transfection reagent.

It is worthy to note that, in another embodiment, a hetero-disaccharide containing two different monosaccharide units can be incorporated with the nucleic acid, for example, lactose formed with galactose and glucose via a β(1→4) bond or sucrose formed with glucose and fructose via a α(1→2) bond.

Thereafter, in step S130, the above transfection reagent containing the nucleic acid and the disaccharide is applied to cells to transfect the nucleic acid into the cells. The cells referred herein can be an in vitro cells or an in vivo cells. For example, in step S130, a cell culturing method is used to transfect the nucleic acid into a cell line or an intravenous injection, intratumoral injection, or intramascular injection type of delivery method is used to perform in vivo transfection. When an in vitro transfection is performed with the transfection reagent of the invention, the concentration of the disaccharide in the transfection reagent is between about 10 mM to about 300 mM. When an in vivo transfection is performed with the transfection reagent of the invention, the concentration of disaccharide is about 15 mg/kg body weight to about 1000 mg/kg body weight. In other words, when the transfection reagent is applied in an in vivo transfection, the amount of the disaccharide used is adjusted according to the weight of the animal. In one embodiment, the transfection reagent is applied to a hamster. When the average weight of a hamster is 25 g and the volume of the transfection reagent being injected into the hamster is about 200 μl, the concentration of the disaccharide in the transfection reagent is about 5 mM to about 330 mM.

The method used in applying the transfection reagent to cells includes a vector-free electroporation system or a naked nucleic delivery system, or using a vector to deliver the nucleic acid into the cells. In one embodiment, if a vector is used to deliver the nucleic acid into the cells, the vector must be incorporated into the transfection reagent prior to the application of the transfection reagent on the cells. The vector includes, for example, a viral vector or a non-viral vector. In general, a non-viral vector is formed by synthetic compounds, such as lipids and polymers.

The lipid-based vectors include cationic lipids, such as non-cholesterol-based cationic lipids and cholesterol-based cationic liquids. The positively charge cationic lipids can bond with the negatively charged phosphate backbone of the nucleic acid to form tight and uniform particles, which can facilitate transfection. The above non-cholesterol-based cationic lipids include, for example, (N-[1-(2,3-dioleyloxy)propyl]-N,N,N-trimethylammonium chloride (DOTMA) or the derivatives thereof obtained from modifying the charge or structure or by altering the lengths of the acyl chain. In one embodiment, the liposomes fabricated using the above non-cholesterol-based cationic lipids or cholesterol-based cationic lipids may selectively contain co-lipids, wherein the co-lipids can be cholesterol or L-α-doleoyl posphatidylethanolamine (DOPE). Moreover, the liposome fabricated using the above cationic lipids may be also combined with polymers to form lipopolyplexs. The polymers that may be combined with liposomes include, for example, cationic polymers, such as protamine, polylysine, histone or adenoviral-derived mu peptide.

On the other hand, the polymers that may be used as a vector include cationic polymers, wherein the mechanism is similar to that of the cationic lipids in which bondings occur between the positively charged polymers and the negatively charged phosphate backbone of the nucleic acid to form tight complexes. In this embodiment, the polymer vector can be polyethyleneimime (PEI), for example.

More particularly, when the above non-viral vector is a lipid-based vector, the disaccharide is, for example, trehalose or cellobiose. On the other hand, when the non-viral vector is a polymer-based vector, the disaccharide is trehalose, for example.

A disaccharide is an effective stabilizer for the lipid membrane and is effective in stabilizing the lipopolyplexs formed with lipids and polymers. Moreover, a disaccharide can strengthen the bonding between the nucleic acid and the polymer. Hence, the transfection efficiency can be improved by incorporating a disaccharide into the transfection reagent. Further, not only a disaccharide can increase the level of transfection efficiency, the toxicity of the transfection agent to the cells can be mitigated. By incorporating dissacharides into the existing transfection agent precludes additional chemical reactions and the subsequent purification or characterization processes.

Although trehalose or cellobiose or lactose is incorporated into a transfection reagent to enhance the transfection efficiency in the aforementioned embodiments, the applications of disaccharides according to this invention are not limited as such. This invention may be embodied in many different forms and should not be construed as limited to the embodiments set forth herein. For example, other disaccharides or a mixture of different disaccharides may be used as an enhancer.

To substantiate the effectiveness of the transfection reagent and the method of enhancing the transfection efficiency of the present invention, the actual application of the transfection reagent and the method of enhancing the transfection efficiency of the present invention using various transfection systems for cell transfection, and the effects of the transfection reagent in in vitro applications or in vivo applications will be described. It should be appreciated that this invention should not be construed as limited to the embodiments set forth herein.

### Using Lipid-Based Vectors for Cell Transfection

In the experiments when a lipid-based vector is used for transfecting cells, DOTAP, DC-Chol may be used as the cationic lipids, and cholesterol or DOPE is added as the co-lipids. In the following experiments, different disaccharides are incorporated into a DNA-containing or cationic liposomes-containing transfection reagent, wherein the cationic liposomes include DOTAP, DOTAP/Chol, DOTAP/DOPE or DC-Chol/DOPE, for example. Moreover, in the following experiments, different disaccharides are incorporated with DOTAP, protamine and DNA to form lipopolyplex (represented as LDP hereinafter) and the effects of disaccharides on cell transfection are observed.

### Experiment I

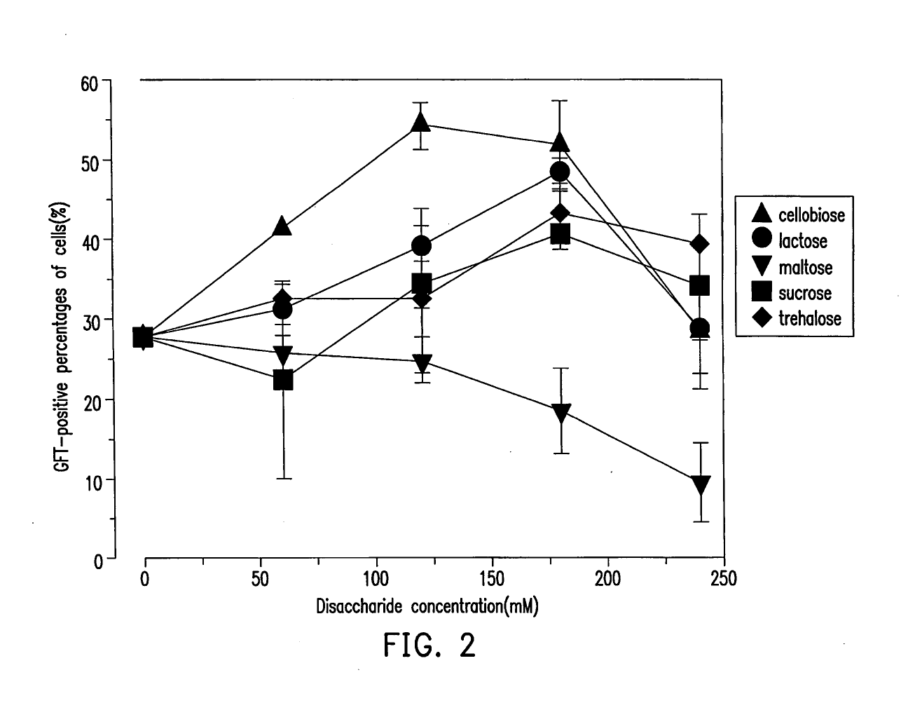

FIG. 2 present the effects of disaccharides on the transgene expression of cultured Chinese hamster ovary (CHO) cells subjected to an in vitro transfection of DNA with a transfection reagent containing DOTAP and various disaccharides. The x-axis in FIG. 2 refers to the disaccharide concentration in the transfection reagent, and the y-axis in FIG. 2 represents the GFP (green fluorescent protein)-positive percentage of the transfected CHO cells measured by a flow cytometer. The GFP-positive percentage of the CHO cells is used as an indicator of transfection efficiency. In the experiments shown in FIG. 2, various concentrations of cellobiose, lactose, sucrose, trehalose and maltose are respectively incorporated into a transfection reagent containing pEGFP-C1 plasmid and DOTAP liposomes, and in vitro transfections are performed with the transfection reagents containing different disaccharides. The GFP-positive percentage of CHO cells is then detected for each transfection reagent sample. As shown in FIG. 2, the incorporation of a disaccharide into the transfection reagent affects the GFP-positive percentage of the transfected CHO cells. In other words, the incorporation of a disaccharide into the transfection reagent affects the transfection efficiency. Moreover, the changes in transfection efficiency are related to the type and the concentration of the disaccharide in the transfection reagent. The transfection efficiency increases significantly when an appropriate amount of cellobiose, lactose, sucrose, trehalose or maltose is incorporated into the transfection reagent. More particularly, the transfection efficiency increases from 28% to more than 40% at 180 mM of lactose, sucrose or trehalose in a transfection reagent. The transfection efficiency is doubled when 120 mM of cellobiose is incorporated into a transfection reagent.

### Experiment 2

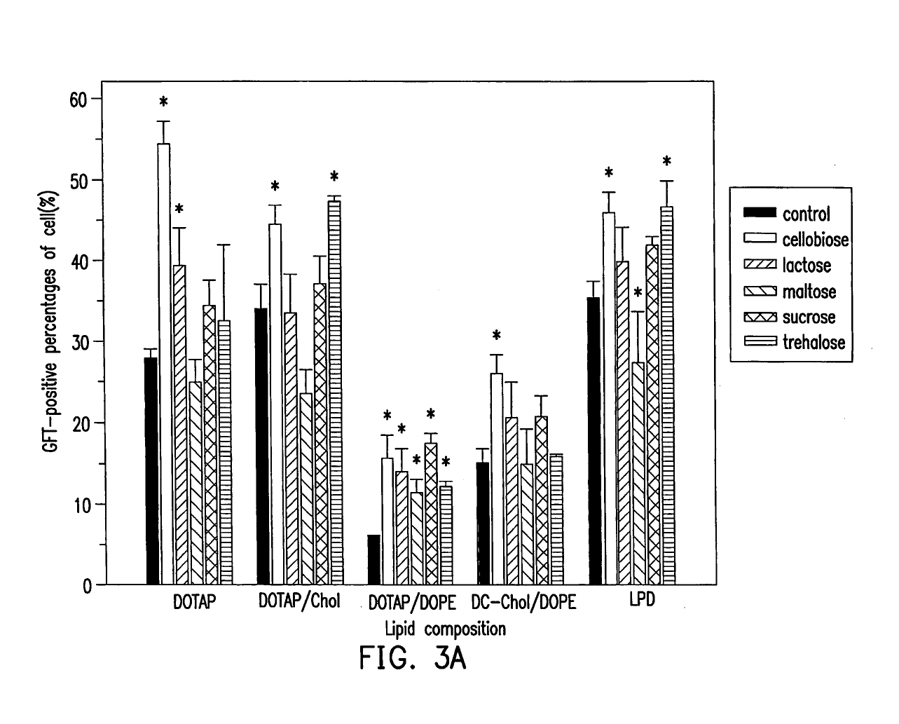

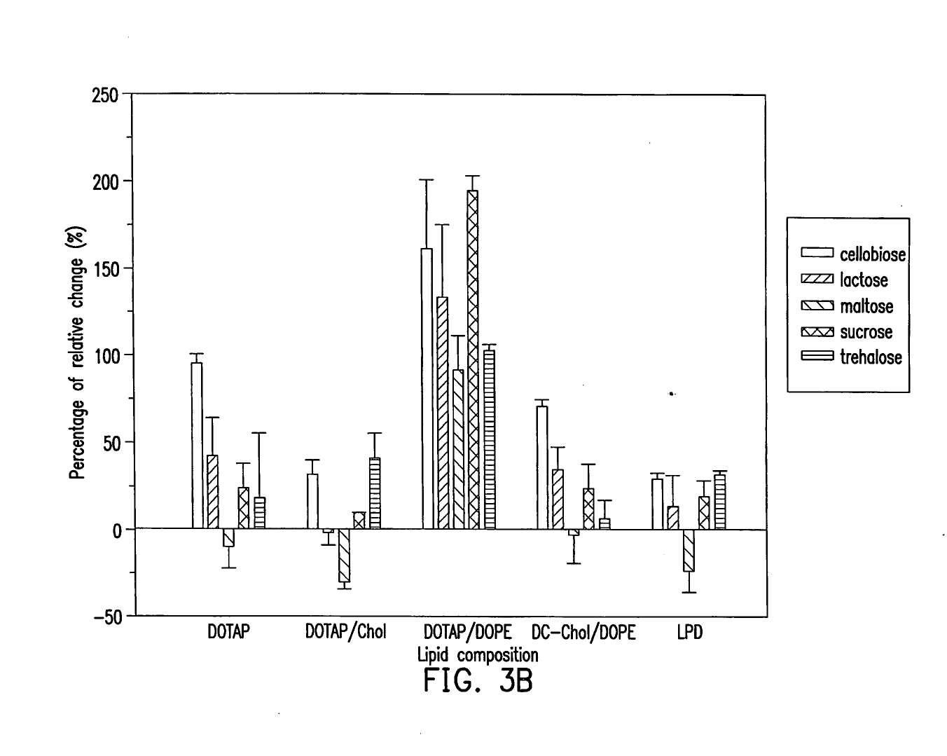

FIGS. 3A and 3B present the effects of disaccharides on the transgene expression of cultured Chinese hamster ovary (CHO) cells subjected to an in vitro transfection of DNA with a transfection reagent containing different lipid-based vectors plus different disaccharides. The x-axis in FIGS. 3A and 3B represents the lipid composition, and the y-axis represents the GFP-positive percentage of the transfected CHO cells, which is used as an indicator for transfection efficiency. In the experiments shown in FIGS. 3A and 3B, DOTAP, DOTAP/Chol, DOTAP/DOPE, DC-Chol/DOPE or LPD applied as a vector is incorporated into a pEGFP-C1 plasmid-containing transfection reagent. Thereafter, 120 mM of various disaccharides is incorporated into the transfection reagent, and in vitro cell transfection is performed followed by detecting the cell percentage of GFP expression. As shown in FIGS. 3A and 3B, irrespective of DOTAP, DOTAP/Chol, DOTAP/DOPE, DC-Chol/DOPE or LPD, the incorporation of cellobiose, lactose, sucrose or trehalose significantly enhances the transfection efficiency of cells.

### Experiment 3

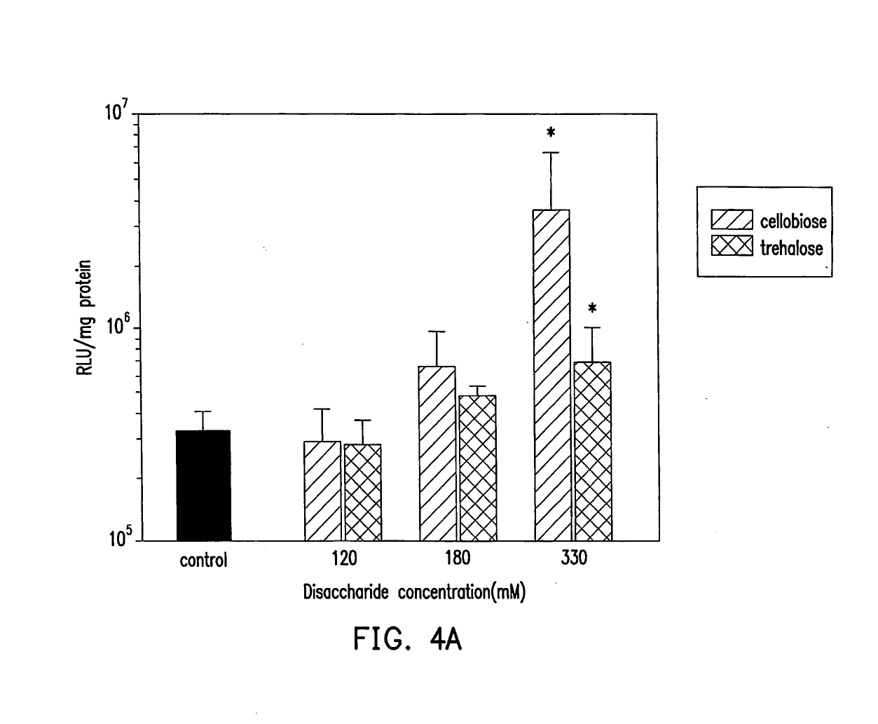

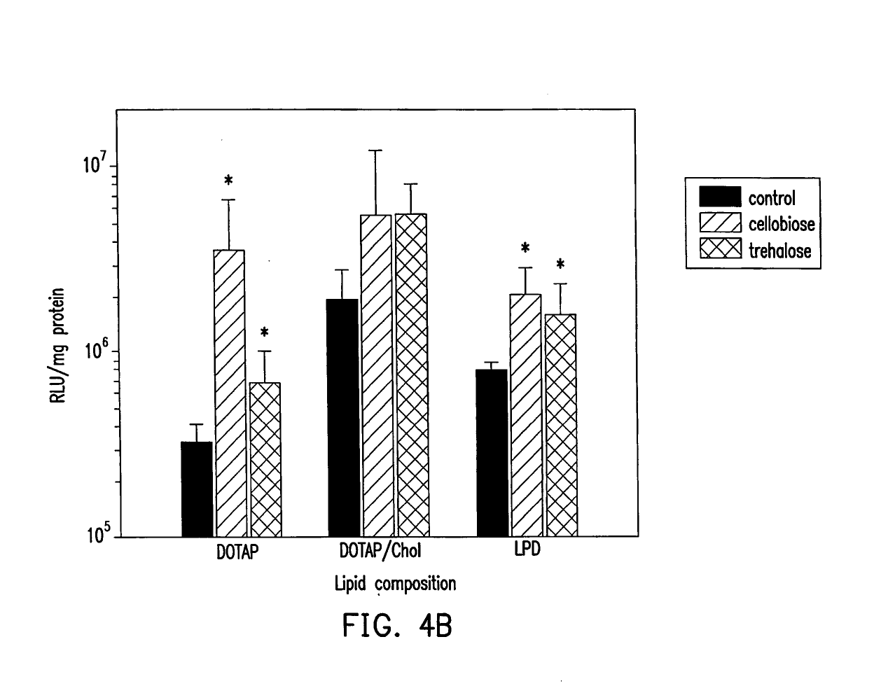

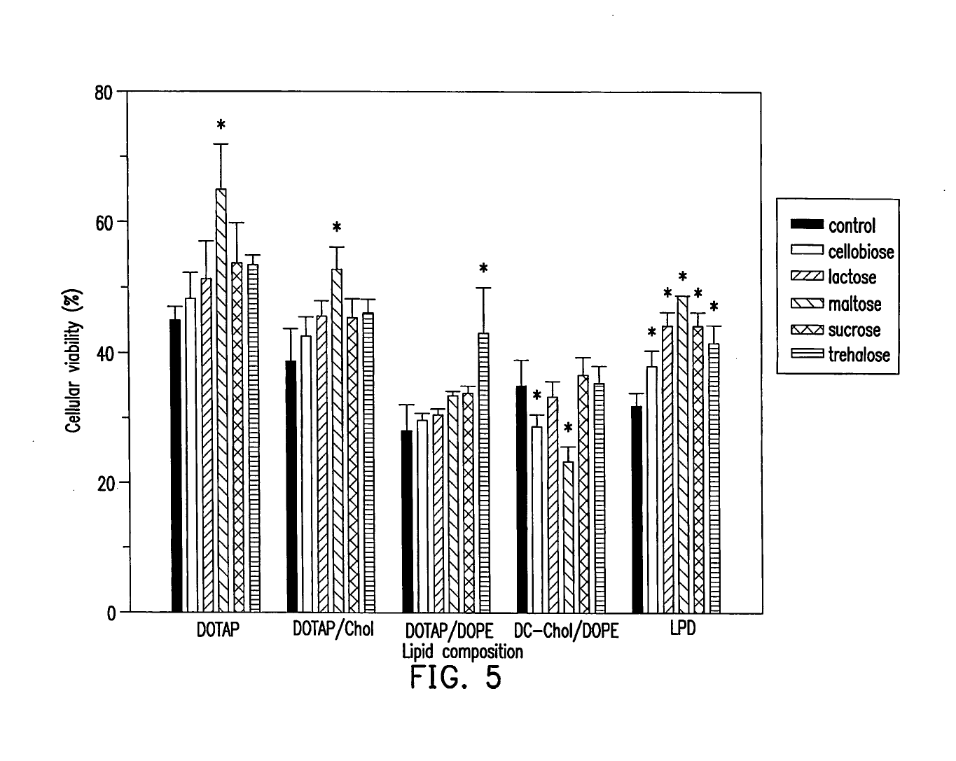

FIG. 4A presents the effects of disaccharides on the transgene expression after intravenous injection of DOTAP-based vectors with various disaccharides by way of mouse tail vein. Various concentrations of cellobiose and cellobiose are respectively incorporated into the transfection reagent containing the gWiz-luciferase plasmid and DOTAP liposomes. The transfection reagent is administered via intravenous injection through the tail vein of a 4-6 weeks old ICR (CD-1) hamster. The hamster is sacrificed 24 hour after the intravenous injection, and the activity of luciferase in the lung tissues of the hamster is observed. The x-axis in FIG. 4A represents the disaccharide concentration in the tranfection reagent, and the y-axis in FIG. 4 represents the luminescent intensity, measured by a luminometer and expressed as relative light unit (RLU) per mg of lung tissue protein. The luminescent intensity is used as an indicator of transfection efficiency. As shown in FIG. 4A, the presence of cellobiose and trehalose can effectively increase the luminescent intensity in the lung tissue. At a concentration of 180 mM, both cellobiose and trehalose can increase the luminescent intensity significantly. As the concentration of cellobiose and trehalose in the transfection reagent increases to 330 mM, the luminescent intensity increases noticeably; more particularly, cellobiose could increase the luciferase expression of lung by more than 10-fold. Accordingly, cellobiose and trehalose can improve the efficiency of cell transfection in vivo, and the transfection efficiency increases as the concentration of the disaccharide increases.

FIG. 4B presents the effects of disaccharides on the transgene expression after intravenous injection of different lipid-based vectors with various disaccharides by way of mouse tail vein. DOTAP, DOTAP/chol and LPD are respectively added as a vector into the tranfection reagent containing gWiz-luciferase plasmid. A concentration of 330 mM cellobiose or trehalsoe is incorporated into the transfection reagent. The transfection reagent is then intravenously injected into the animal for cell transfection. The x-axis in FIG. 4B represents the type of the lipid-based vector, and the y-axis in FIG. 4B represents the luminescent intensity, measured by a luminometer and expressed as relative light unit (RLU) per mg of lung tissue protein. As shown in FIG. 4B, irrespective of DOTAP, DOTAP/Chol and LPD being used as a vector, trehalose and cellobiose are found to result in a significant increase in transfection efficiency.

### Experiment 4

FIG. 5 presents the effects of disaccharides on the cellular cytotoxicity of the in vitro cultured CHO cells transfected with transfection reagents containing lipid-based carriers. The x-axis in FIG. 5 represents the various disaccharides and the y-axis represents the cellular viability analyzed by the MTT assay. As shown in FIG. 5, the viability of the CHO cells that have not been transfected is set at 100%. As shown in FIG. 5, when using DOTAP, DOTAP/Chol, DOTAP/DOPE or LPD as a vector, the incorporation of cellobiose, lactose, maltose, sucrose or trehalose respectively improve the cellular viabilities at different degrees. In other words, the incorporation of disaccharides into a transfection reagent effectively mitigates the toxic effect of the transfection reagent on cells.

### Using a Polymer-Based Vector For Cell Transfection

In the following experiments, a polyethyleneimine (PEI)-based vector is used to perform cell transfection. Further, various disaccharides are incorporated into the transfection reagent containing DNA and the vector, and the effects of disaccharides on cell transfection are observed.

### Experiment 5

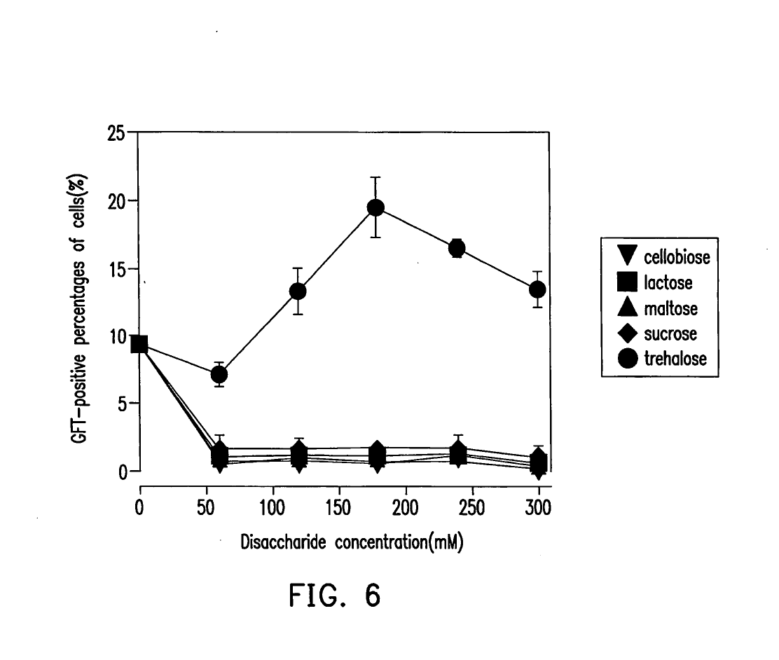

FIG. 6 presents the effects of disaccharides on the transfection efficiency of in vitro cultured CHO cells that are transfected with transfection reagents containing PEI and different disaccharides. The x-axis in FIG. 6 represents the concentrations of different disaccharide in the transfection reagent, and the y-axis represents the GFP expression percentage of the transfected CHO cells detected by a flow cytometer. The GFP expression percentage of the transfected CHO cells is used as an indicator of transfection efficiency. As shown in FIG. 6, the charge ratio of PEI to DNA in the transfection reagent is maintained at 9:1, and various concentrations of cellobiose, lactose, maltose, sucrose and trehalose are incorporated into the transfection reagent. The effect of disaccharides in the transfection reagent on transfection efficiency is then analyzed. According to the experimental results, the incorporation of trehalose into the transfection reagent significantly increases the GFP expression percentage. In other words, the cell transfection efficiency is enhanced. More particularly, at a trehalose concentration of 180 mM in the transfection reagent, the GFP expression percentage increases to about 20%, which is about one fold higher than that in the absence of disaccharides.

### Experiment 6

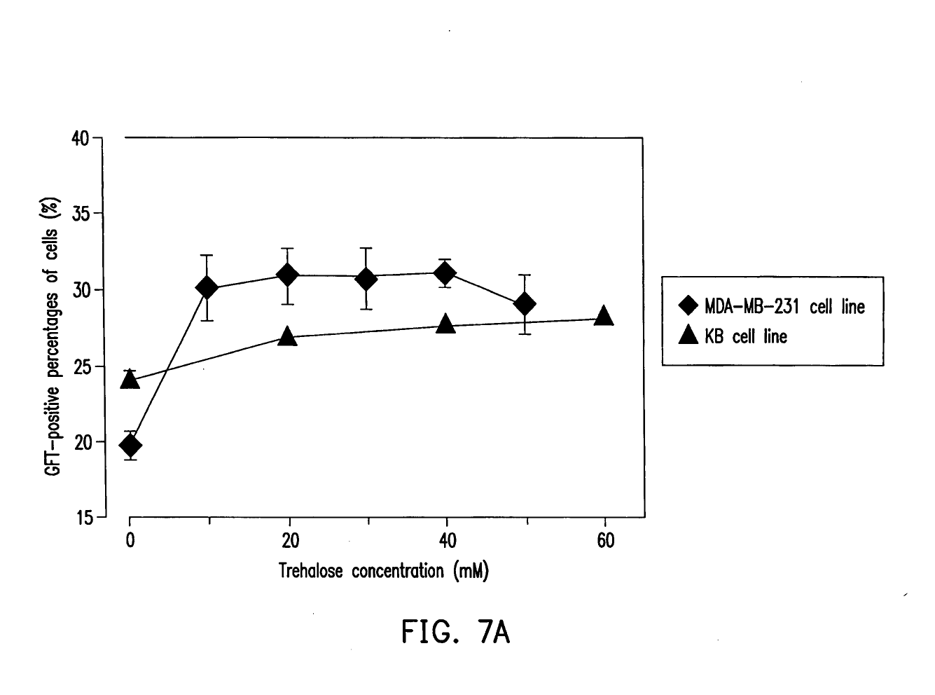

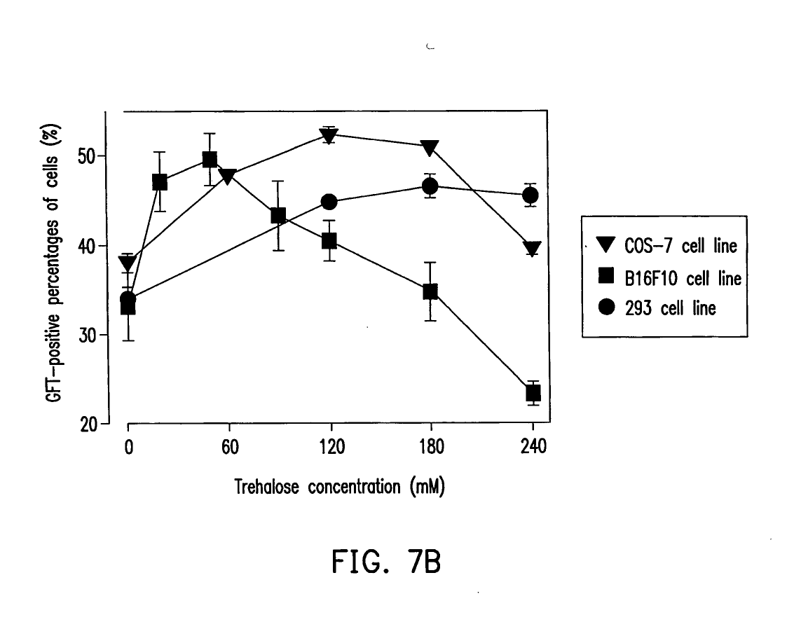

FIGS. 7A and & 7B show the optimal trehalose concentrations for enhancing the transfection expression mediated by DNA-PEI complexes in different cell lines. The x-axis in FIGS. 7A and 7B 6 represents the concentrations of trehalose in the transfection reagent, and the y-axis represents the GFP expression percentage of the cells as an indicator of transfection efficiency The transfection procedures in these experiments of different cell lines are identical to those described previously for CHO cells in experiment 5. As shown in FIGS. 7A and 7B, the addition of trehalose into a transfection reagent significantly increases the transfection efficiency of each cell line, including MDA-MB-231, KB, COS-7, B16F10 and the 293. Moreover, the extents of enhancement on the transfection efficiency depend on the cell lines. More particularly, at a trehalose concentration of 180 mM in the transfection reagent, the percentages of GFP expressed cells are enhanced by 37% and 32%, respectively, for the COS-7 cells and the 293 cells. However, a low trehalose concentration of 20 mM effectively enhances the percentage of GFP positive cells for the MDA-MD-231, B16F10, and KB cell lines by about 50%, 42% and 13%, respectively.

### Experiment 7

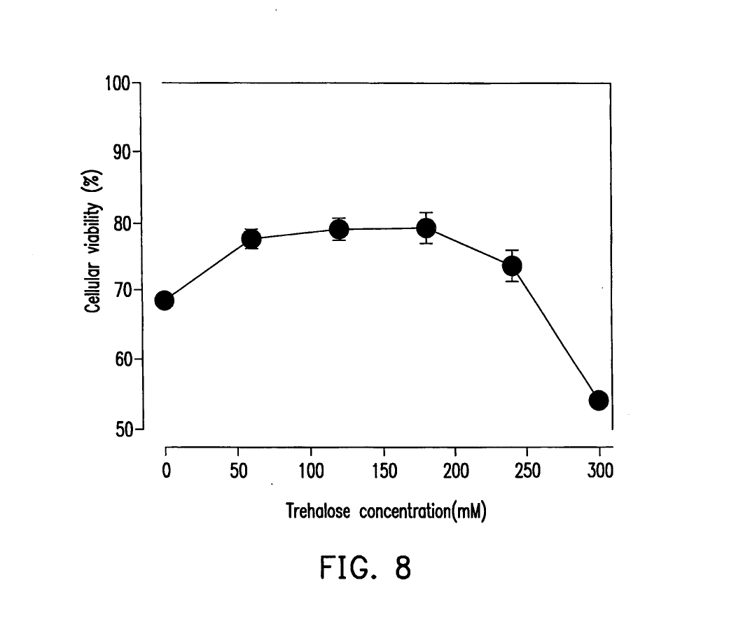

FIG. 8 presents the effects of trehalose on the cellular cytotoxicity of the in vitro cultured CHO cells transfected with transfection reagents containing DNA-PEI complexes. The x-axis in FIG. 5 represents different trehalose concentrations and the y-axis represents cellular viability analyzed by the MTT assay. In the experiments, the PEI to DNA charge ratio in the transfection reagent is maintained at 9:1, and the viability of nontransfected CHO cells is set at 100%. As shown in FIG. 8, the incorporation of trehalose into the transfection reagent increases the cellular viability. In other words, the incorporation of trehalose mitigates the toxic effect of the transfection reagent.

### Cell Transfection Using Naked Nucleic Acid Delivery System

The following experiments are conducted using naked nucleic acid delivery system for directly transfecting plasmid DNA into the cells of animals, and additional viral or non-viral vector is precluded. The transfection reagent is incorporated with various concentrations of disaccharides as an enhancer, and the effects of disaccharides in the naked nucleic acid delivery system on the cell transfection are observed.

### Experiment 8

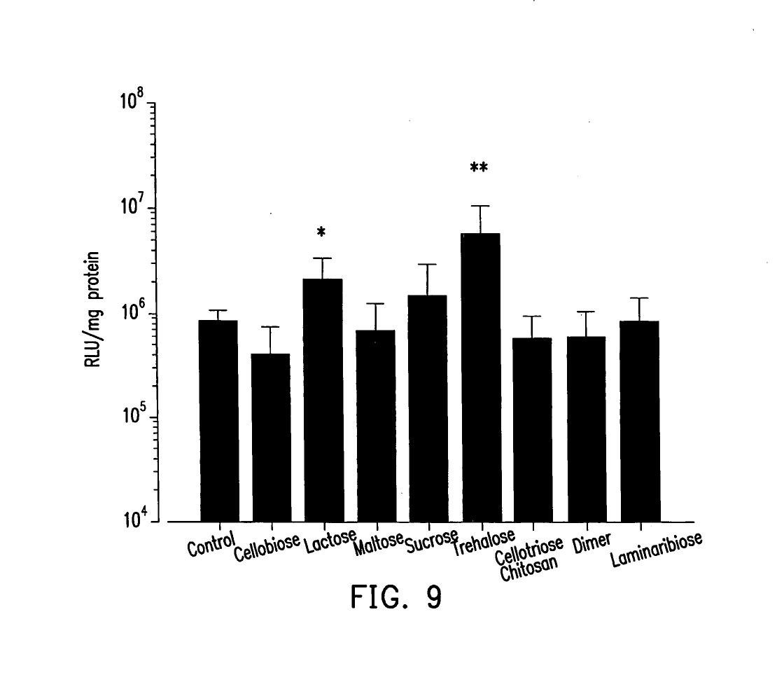

FIG. 9 presents the effects of various disaccharides on the transgene expression of mouse muscles after direct intramuscular injection of naked plasmid. The transfection procedures in these experiments are identical to those described previously in experiment 3. The major differences between the two experiments are the vector applied and the method of transfection as well as the administration route into the body of the hamster. The x-axis in FIG. 9 represents the types of disaccharides, and the y-axis represents the luminescent intensity, measured by a luminometer and expressed as relative light unit (RLU) per mg of muscular tissue protein, wherein the luminescent intensity is used as an indicator to evaluate the transfection efficiency. As shown in FIG. 9, when comparing to the control group in the absence of disaccharides in the transfection reagent, the incorporation of 10 mM of trehalose and lactose into the transfection reagent results in a higher luminescent intensity. In other words, trehalose and lactose significantly increase the efficiency in in vivo transfection of muscles by direct injection of naked plasmid.

### Experiment 9

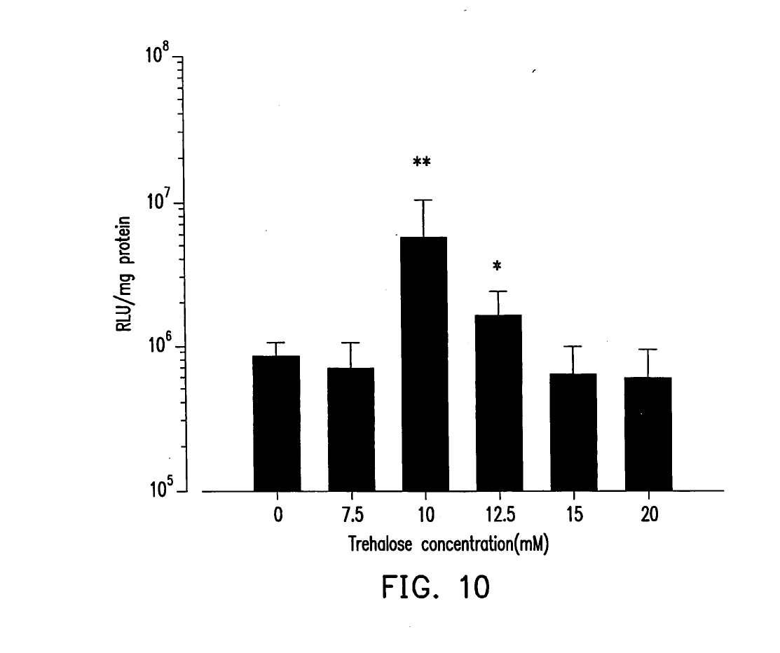

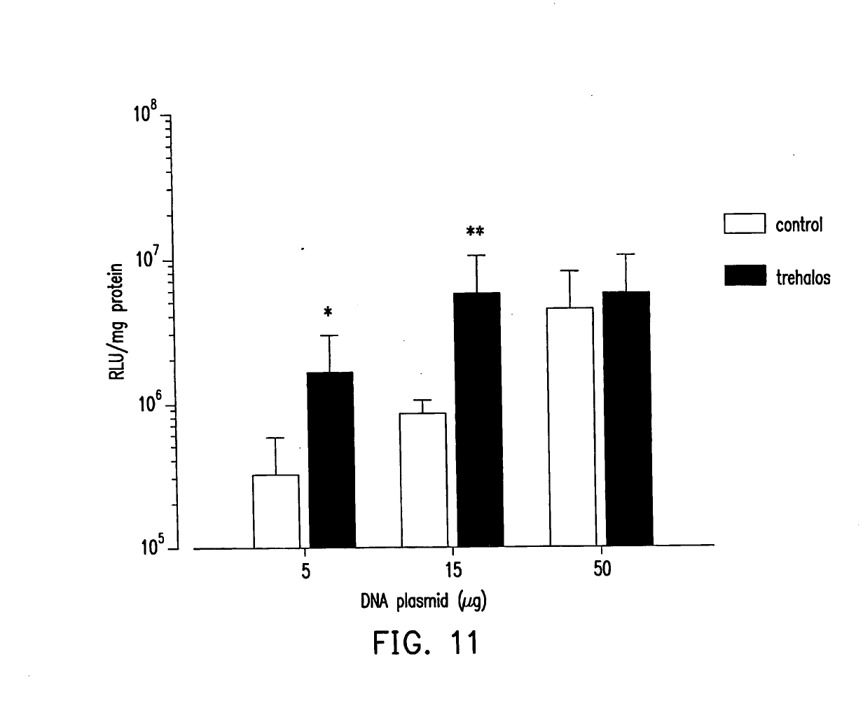

FIG. 10 presents the effects of trehalose concentrations on the transgene expression of mouse muscles after direct intramuscular injection of naked plasmid. The x-axis in FIG. 10 represents the concentrations of trehalose in the transfection reagent, and the y-axis represents the luminescent intensity, measured by a luminometer and expressed as relative light unit (RLU) per mg of muscular tissue protein. The luminescent intensity is used as an indicator to evaluate transfection efficiency. According to the results of in vivo transfection of DNA into muscles using a naked nucleic acid delivery system, the transfection efficiency increases significantly when the concentration of trehalose in the transfection reagent is between about 10 mM to 12.5 mM.

Experiment 11 presents the effects of gene dosage on the transgene expression of mouse muscles in the presence of trehalose after direct intramuscular injection of naked plasmid. The x-axis in FIG. 11 represents the amount of DNA plasmid, and the y-axis represents the luminescent intensity, measured by a luminometer and expressed as relative light unit (RLU) per mg of muscular tissue protein, wherein the luminescent intensity is used as an indicator to evaluate the transfection efficiency. As shown in FIG. 11, when the same amount of plasmid DNA is used, the transfection efficiency of a transfection reagent with the addition of 10 mM of trehalose is significantly higher than that of a control group without the addition of trehalose. More particularly, the transfection efficiency of a transfection reagent with the addition of trehalose and 15 μg of DNA plasmid is similar to that of a transfection reagent with no trehalose and 50 μg of DNA plasmid. Hence, the application of trehalose can reduce the amount of nucleic acid used while a desirable transfection efficiency is resulted.

### Experiment 11

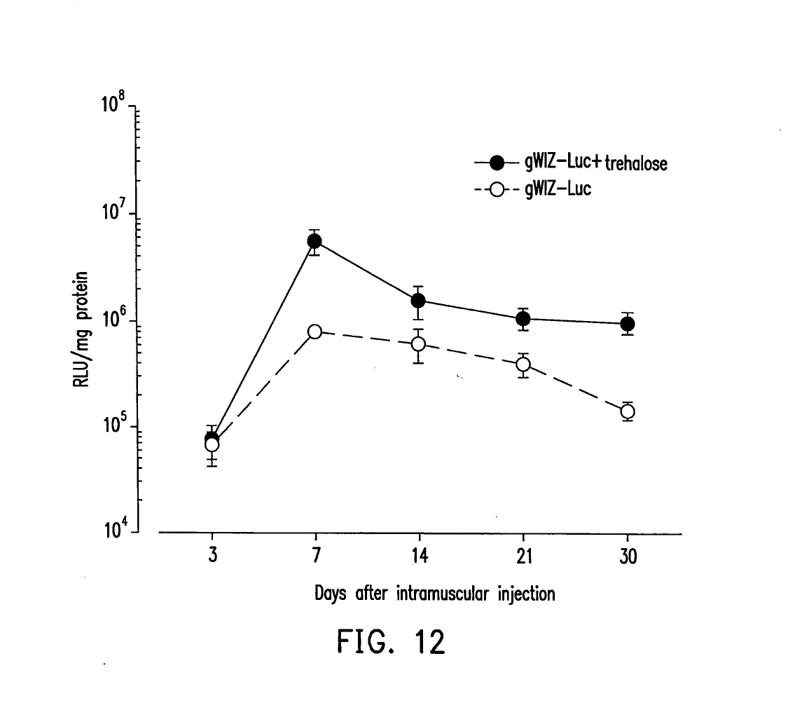

FIG. 12 presents the effects of the trehalose on the durations of transgene expression of mouse muscles after direct intramuscular injection of naked plasmid. The x-axis in FIG. 12 represents the days after the intramuscular injection, and the y-axis represents the luminescent intensity, measured by a luminometer and expressed as relative light unit (RLU) per mg of muscular tissue protein, wherein the luminescent intensity is used as an indicator to evaluate the transfection efficiency of gWiz-luciferase plasmid. The experimental results indicate that the incorporation of trehalose results in higher luminescence intensities for a longer period of time. In other words, trehalose might extend the durations of nucleic acid remaining inside the cells, and therefore prolonged the durations of transgene expression.

In accordance to the above experimental results, the incorporation of disaccharides in a transfection reagent provides better in vitro and in vivo transfection efficiencies. With the combination of disaccharides and a small amount of nucleic acid in a transfection reagent, not only the transfection efficiency is enhanced, the amount of nucleic acid used is reduced. Moreover, the above experimental results confirm that the application of a tranfection reagent containing disaccharides can increase the transfection efficiency when the different vectors or delivery methods are used. Additionally, based on the above experimental results, not only the incorporation of disaccharides into a transfection reagent enhances tranfection efficiency, the cytotoxicity of the transfection reagent is lowered and the viability of the transfected cells is greatly improved.

In accordance to the transfection reagent and the method of enhancing transfection efficiency of the present invention, at least the following advantages are provided.

1. The incorporation of disaccharides with nucleic acid as a tranfection reagent can improve the efficiencies of both in vitro transfection and in vivo transfection. Moreover, the durations of the nucleic acid remaining in the cells can also be extended.

2. The present invention can provide desirable transfection efficiency even when the amount of nucleic acid used is lowered.

3. The present invention allows the application of different transfection techniques without additional complicated procedures. Hence, aside from improving the transfection efficiency, cell transfection can be facilitated.

4. The present invention further lowers the cytotoxicity of transfection reagent on cells.

The present invention has been disclosed above in the preferred embodiments, but is not limited to those. It is known to persons skilled in the art that some modifications and innovations may be made without departing from the spirit and scope of the present invention. Therefore, the scope of the present invention should be defined by the following claims.

What is claimed is:

1. A transfection reagent, comprising:
a nucleic acid; and
a disaccharide, wherein the disaccharide is formed with two identical monosaccharide units.

2. The transfection reagent of claim 1, wherein the disaccharide is trehalose or cellobiose.

3. The transfection reagent of claim 1 further comprising a vector.

4. The transfection reagent of claim 3, wherein the vector is a viral vector.

5. The transfection reagent of claim 3, wherein the vector is a non-viral vector.

6. The transfection reagent of claim 5, wherein the non-viral vector is a lipid-based vector or a polymer-based vector.

7. The transfection reagent of claim 6, wherein when the non-viral vector is the lipid-based vector, the disaccharide includes trehalose or cellobiose.

8. The transfection reagent of claim 6, wherein when the non-viral vector is the polymer-based vector, the disaccharide includes trehalose.

9. The transfection reagent of claim 1, wherein the transfection reagent is applied in an electroporation system or a naked plasmid delivery system.

10. The transfection reagent of claim 1, wherein the transfection reagent is applicable in an in vitro transfection system or an in vivo delivery system.

11. The transfection reagent of claim 10, wherein when the tranfection reagent is applied in the in vitro transfection system, the concentration of the disaccharide in the transfection reagent is between about 10 mM to 300 mM.

12. The transfection reagent of claim 10, wherein when the tranfection reagent is applied in the in vivo delivery system, a concentration of the disaccharide in the transfection reagent is between about 15 mg/kg body weight to about 1000 mg/kg body weight.

13. A method for enhancing transfection efficiency, the method comprising:
providing a nucleic acid;
incorporating a disaccharide into the nucleic acid as a transfection reagent, wherein the disaccharide is formed with two identical monosaccharide units; and
applying the transfection agent to cells to transfect the nucleic acid into the cells.

14. The transfection reagent of claim 13, wherein the disaccharide is trehalose or cellobiose.

15. The transfection reagent of claim 13 further comprising a vector.

16. The transfection reagent of claim 15, wherein the vector includes a viral vector.

17. The transfection reagent of claim 15, wherein the vector is a non-viral vector.

18. The transfection reagent of claim 17, wherein the non-viral vector is a lipid-based vector or a polymer-based vector.

19. The transfection reagent of claim 17, wherein when the non-viral vector is the lipid-based vector, the disaccharides includes trehalose or cellobiose.

20. The transfection reagent of claim 17, wherein when the non-viral vector is the polymer-based vector, the disaccharides includes trehalose.

21. The transfection reagent of claim 13, wherein the transfection reagent is applied in an electroporation system or a naked plasmid delivery system.

22. The transfection reagent of claim 13, wherein the transfection reagent is applicable in an in vitro transfection system or an in vivo delivery system.

23. The transfection reagent of claim 22, wherein when the tranfection reagent is applied in the in vitro transfection system, the concentration of the disaccharide in the transfection reagent is between 10 mM to 300 mM.

24. The transfection reagent of claim 22, wherein when the tranfection reagent is applied in the in vivo delivery system, a concentration of the disaccharide in the transfection reagent is between 15 mg/kg body weight to about 1000 mg/kg body weight.
